(19) United States
(12) Patent Application Publication
(10) Pub. No.: US 2009/0182291 A1
(43) Pub. Date: Jul. 16, 2009

(54) EYE MEDICAMENT DISPENSER

(76) Inventor: Eran Eilat, Herzliya (IL)

Correspondence Address: FENNEMORE CRAIG, 3003 NORTH CENTRAL AVENUE, SUITE 2600, PHOENIX, AZ, 85012, US

(21) Appl. No.: 12/299,745

(22) PCT Filed: May 10, 2007
(86) PCT No.: PCT/IL07/00565

§ 371 Date: Nov. 17, 2008

Related U.S. Application Data
(60) Provisional application No. 60/799,328, filed on May 11, 2006.

(51) Int. Cl. A61F 9/00

(52) U.S. Cl. 604/290; 604/296

(57) ABSTRACT

A device for applying a medicament to the eye, consisting of a single or pair of eye goggles, into which is sprayed a fine mist or fog of the medicament. The medicament is contained in a canister attached to an aperture in the goggle or goggles, and the mist is generated either by the configuration of the outlet valve, or by means of a baffle disposed within the goggle volume opposite the outlet valve. The container may be a bag-on-valve type of device, with the aerosol propellant outside of the medicament bag, such that the medicament does not come into contact with the propellant. Alternatively, the container may be a metered-dose inhalator type of container operated by manual pressure. Because of the one-way flow operation, and the separation of propellant and medicament, formulation of the medicament with a potentially harmful preservative is not necessary.

### FIELD OF THE INVENTION

The present invention relates to a device and method dispensing eye drops for treating eye disorders, wherein the pharmacologically active agent is administered to a treated eye in a form of fog or mist. The device and method of administering a medicament in this form provide accurate dosage volumes, optimizes the residence time of the medicament on the eye surface, enhance treatment effectiveness, increase compliance and are convenient to use.

### BACKGROUND OF THE INVENTION

Treating the human eye, and in particular applying medicine to the eyeball, is a sensitive and usually difficult task. The most common form of drug delivery is by topical application of the medicament to the eye's surface, e.g., by drops. The eye is uniquely suited to surface administration because drugs can penetrate through the cornea, reach therapeutic concentration levels inside the eye, and exert their beneficial effects. In fact, ninety to ninety five percent of drugs delivered to the eye are currently administered through eye drops. Rarely are drugs for the eye administered orally or by injection, either because they reach the eye in too low a concentration to have the desired pharmacological effect, or because their use is complicated by significant systemic side effects.

Topical eye drops, though effective, have several drawbacks. When an eye drop is instilled in the eye, it overfills the conjunctival sac, the pocket between the eye and the lids, causing a substantial portion of the drop to be lost due to overflow from the lid margin onto the cheek. In addition, a substantial portion of the drop remaining on the ocular surface is drained into the nasolacrimal duct, thereby diluting the concentration of the drug. Not only is this portion of the drug dose lost before it can cross the cornea, but this excess drug is carried into the nose and throat where it may be absorbed into the local or general circulation, leading to systemic side effects, sometimes serious.

To compound the problems described above, patients often do not use their eye drops as prescribed. Often, this poor compliance is due to an initial stinging or burning sensation caused by the eye drop. Certainly, instilling eye drops in one's own eye can be difficult, in part because of the normal reflex to protect the eye. Therefore, sometimes one or more drops miss the eye. Older patients may have additional problems instilling drops due to arthritis, unsteadiness, and decreased vision, and pediatric and psychiatric patient populations pose difficulties as well. Bottled eye medication need refrigeration after being opened, hence the cold drops adding to the discomfort. Finally, most currently used formulations in ophthalmology need preservatives, mainly BAK, a substance known to be toxic to the cornea.

There have been several developments to assist patients in applying medication to the eye. These prior developments focus on easing the application of a drop of liquid medication onto the eye's surface. U.S. Pat. No. 6,610,036 provides an eye drop dispensing device that includes a trough member with a space to receive a cartridge housing enclosing a collapsible bag containing an ophthalmic liquid and comprising a spring finger applying pressure to the bag. It allows for spring-powered pump to spray a predetermined quantity of an ophthalmic liquid into a user's eye.

U.S. Pat. No. 6,336,917 provides an eye mist apparatus for treatment of an eye having an outer housing that contacts the bony orbit surrounding the eye. An inner housing, concentrically disposed within the outer housing contacts the eyelid, pushing the eyelid back, exposing the eye. One end of the inner housing is adapted to receive a dispenser for administering a metered spray of medicine, powdered or liquid, or lavage to the eye as it is held open by the ocular treatment apparatus. The dispenser nozzle is equipped with a contrasting mark, which provides a target for the eye to focus on ensuring the eye is optimally positioned to receive the metered spray. The spray is described as being directed straight onto the anterior surface of the eyeball, so that none of the medication is wasted. However, it is possible that delivery directly onto the eyeball surface may cause blinking reflex and discomfort to some users.

U.S. Patent Application Publication No. 2005/0255144 provides articles and methods for drug delivery by way of hydrogel for treatment of dry eye conditions. It allows for passive transference of drug from a dilute solution into the hydrogel. The hydrogel, when placed in contact with the eye, delivers the drug. The delivery of the drug is sustained over an extended period of time.

U.S. Patent Application Publication No. 2006/0020253 provides an implantable device including a body defining a reservoir, and a head provided at the end of the body and closing the reservoir. The head is made from a permeable material adapted to release the medication from the reservoir to an implant site at a determinable rate.

U.S. Pat. No. 5,893,515 provides a device for creating and applying mist, vapors or sprays from a liquid. The device contains a spinning rotor within a mist chamber. The rotor has inner walls that taper conically outwardly from the open bottom of the rotor to a hole near the top of the rotor. Liquid is pumped by a finger actuated pump from a cartridge module into a bowl surrounding the bottom end of the rotor. A spray is created as the liquid is formed into droplets as it passes through the hole in the rotor under centrifugal force.

The problem with devices of this type is they require a significant amount of manual dexterity to position the apparatus over the eye, and a steady hand to maintain the apparatus over the eye. Additionally problematic, the above devices require patients to tilt their head back so the medicine bottle is in a vertical position, allowing the drop to fall into the eye by force of gravity. This is often difficult for elderly arthritic patients who experience difficulty moving the neck, or have weak and unsteady hands. Furthermore, the medication stream is directed straight onto the eyeball, such that it may often cause a blinking reflex, which prevents the medicament to reach the eye surface or may not be comfortable for all users.

U.S. Patent Application Publication No. 2002/0124843 provides an apparatus and a method for delivering medicine for one or more eyes. The apparatus includes goggles worn around the eyes. An atomizer nebulizes medicine into a medicine carrying fog such that the fog discharges from the fog outlets to deliver medicine to one ore more eyes. However the apparatus requires a comparatively complex system for nebulization of the medicine, such as an ultrasonic atomizer. Additionally, according to those embodiments of this invention where a propellant is used, the propellant does not appear to be separated from the medicine. Furthermore, it would appear from the embodiments shown that the medicament is stored in a freely fillable container, such that sterility may be difficult to maintain.

U.S. Pat. No. 6,962,151 discloses a nebulizer for atomizing liquid solutions (i.e. convert to aerosol). The nebulizer includes an aerosol generator that atomizes the liquid through a vibrating diaphragm into particle sizes that are efficiently delivered to the treated area. This nebulizer is currently commercialized under the trade name eFlow®. Classic jet and ultrasonic nebulizers have the disadvantage of potentially denaturizing the active agent by high shear forces (jet and ultrasonic nebs) and temperature increase (ultrasonic nebs). eFlow® incorporates a “gentle” aerosolization mechanism that minimizes exposure of the drug to shear stresses by reducing the shear stresses and the residence time in the shear fields and does not heat the liquid formulation. U.S. Pat. Nos. 5,152,456; 5,261,601, and 5,518,179 disclose further aspects of the eFlow® technology.

PCT Published Application No. WO2006/082588 describes a device for ophthalmic administration of pharmaceutical ingredients configured to direct a mist of the pharmaceutical composition at the eye. In this application, there is recognition of the need for maintaining sterility of the device. This is achieved by sterilizing the skin contact surface with the device between individual administrations. As stated therein, a device that is quick and easy to sterilize allows high throughput and safe ophthalmic administration of a pharmaceutical composition, for example in a hospital or clinic where many patients may be treated with one device, or in high-throughput situations, for example when desired to treat a population for an epidemic or endemic condition or to inoculate or immunize a population. One embodiment incorporates a self-sterilization device, in the form of an element radiating the contact surface of the device. However, in this application, other than by the assumed use of preservatives, no consideration appears to have been made for the need to ensure that the pharmaceutical composition itself remains sterile, especially in the typical domestic setting of prolonged sequential use of the device by a single person.

Thus, there remains a long felt, significant need for an ocular treatment apparatus that does not require extensive manual dexterity to position and operate, simplifying the application of ophthalmic liquids to the eye. The apparatus should not have any potentially damaging projections and somebody with a weak or unsteady hand should be able to operate the apparatus with ease, and without the potential for injury. There is also a need for an ocular drug delivery system that is capable of dispensing medicine in the eye in an effective, accurate, measured and well-tolerated manner, which is meant to include toleration of the physical application process itself. The system should also be such as to preferably ensure the sterility of the ophthalmic liquid, thus enabling the use of an ophthalmic liquid without the addition of any preservative. It is to be understood that the term “ophthalmic liquids” as used within this application, and as claimed, is meant to include any liquid in the form of a solution, suspension or emulation intended for treatment of the eye or treatment of it surrounding tissues, such as in eyelid disease, or even water. Treatment can be understood to mean therapeutic treatment, preventive treatment or palliative treatment.

The disclosures of each of the publications mentioned in this section and in other sections of the specification, are hereby incorporated by reference, each in its entirety.

### SUMMARY OF THE INVENTION

This invention seeks to provide, according to a first preferred embodiment, a device and method for delivery of a medicament or ophthalmic solution to the eye in the form of a mist or fog made up of nano-particles, applied to the eye region by means of a goggle or pair of goggles into which the mist or fog is directed.

A number of features of the device provide it with advantages over prior art devices. One specific requirement of the device is that the ophthalmic fluid be maintained in a sterile manner, even during prolonged sequential use of a single medicament lot in the device. This aspect of the present device has the important ramification that in a device where sterility is maintained, there is no need for the ophthalmic solution to contain a preservative additive, some of these preservatives being known to have potentially harmful effects on the eye. According to a preferred embodiment of the present claimed invention, this feature is achieved by supplying the ophthalmic solution in a hermetically sealed flexible bag, integrally attached to a dispensing valve, this being known as a bag-on-valve. The solution is forced out of the bag by externally applied pressure. According to one preferred embodiment, this pressure is applied by enclosing the bag in a sealed canister containing a propellant under pressure, such that activation of the dispensing valve allows the propellant to drive a dose of the ophthalmic solution out of the flexible bag. The dose can be arranged to be of predetermined level by use of a metering dose dispenser in the output nozzle, as is known in the art.

Use of such a separate propellant container for the flexible bag is known as the dual-chamber canister technique. This allows the ophthalmic solution to be kept separately from the propellant, such that the propellant does not come into contact with the ophthalmic solution either physically or chemically.

According to another preferred embodiment, the pressure is applied to the flexible bag by means of squeezing action applied by the user. Such an embodiment simplifies the device, without detracting from the advantages presented by the use of the bag-on-valve technology.

According to a third preferred embodiment, the ophthalmic solution can be contained in an inhaler type of pressurized container. Such an embodiment does not have all the advantages of the bag-on-valve embodiments described hereinabove, since the ophthalmic solution is in contact with the propellant, though other above-described advantages are applicable.

The ophthalmic solution can be converted into a mist form either by use of a baffle onto which the solution impinges on entry into the goggle or goggles internal space, or by use of a nebulizing nozzle at the outlet of the actuating valve. Such a nebulizing nozzle can be of passive design, such as a fine mesh membrane placed over the output, in order to maintain simplicity of the device.

A further feature of the claimed device is that the mist is directed into the goggles in such a direction that it does not impinge directly on the eye surface. This is preferably arranged by ensuring that the mist enters the goggles in a direction generally parallel relative to the eye surface, either lateral, medial, superior or inferior. This enables application of the ophthalmic solution without any unpleasant effects to the user, and without any reflex eye-closure effects which would degrade the effectiveness of the application.

The present invention is particularly advantageous in that the medicament does not come in fluid communication with a propellant. Furthermore the system and the method of administering a medicament in this form provide accurate dosage volumes, increase the residence time of the medicament in the eye, enhance treatment effectiveness, increase compliance and may generally be more convenient to use than currently available eye medication delivery systems.

According to this invention the goggle/s enclosing one or both eyes are filled with the produced mist. The eye receives a medication carried in a moist mist or fog having extremely fine, cloud-like particles contained by the goggle/s, in the closed space between the goggles and the area in front of the eye(s). The device and method of this invention can be applied to treat the outside of the eyes for such infections as conjunctivitis, blepharitis, keratitis sicca and the like and for allergic conditions, and to provide intraocular treatment with drugs absorbed through the cornea, conjunctiva and sclera for other eye diseases.

All of the above options enable pre-determining of the amount to be dispensed for different medications, each as specified by their respective labeling, as is known in the art as used in meter dose inhalators.

There is thus provided in accordance with a preferred embodiment of the present invention, a device for applying an ophthalmic liquid to at least one eye of a subject, the device comprising:
(a) a flexible bag containing the ophthalmic liquid,
(b) an output valve assembly at one extremity of the bag,
(c) an outer container enclosing the flexible bag, the outer container containing a propellant gas under positive pressure, such that ophthalmic liquid is ejected from the flexible bag when the valve assembly is activated,
(d) at least one eye goggle having at least one opening in its rim, the outer container being disposed relative to the at least one goggle such that the ejected ophthalmic liquid is directed through the at least one opening into the at least one goggle, and
(e) a nebulizing element configured to disperse the ophthalmic liquid in the form of a mist within the at least one eye goggle.

There is further provided in accordance with yet another preferred embodiment of the present invention, a device for applying an ophthalmic liquid to at least one eye of a subject, the device comprising:
(a) a flexible bag containing the ophthalmic liquid,
(b) an output valve assembly at one extremity of the bag,
(c) at least one eye goggle having at least one opening in its rim, the outer container being disposed relative to the at least one goggle such that the ejected ophthalmic liquid is directed through the at least one opening into the at least one goggle, and
(d) a nebulizing element configured to disperse the ophthalmic liquid in the form of a mist within the at least one eye goggle,
   wherein the flexible bag is configured such that when squeezed, a dose of the ophthalmic liquid is dispersed in the form of a mist within the at least one goggle.

In accordance with still another preferred embodiment of the present invention, there is provided a device for applying an ophthalmic liquid to the eye of a subject, comprising:
(a) a rigid container containing the ophthalmic liquid and a propellant gas,
(b) an output valve at a first extremity of the rigid container,
(c) a housing in which the rigid container is disposed, such that pressure on a second extremity of the container activates the output valve and ejects ophthalmic liquid from the rigid container,
(d) at least one eye goggle having at least one opening in its rim, the housing being disposed relative to the at least one goggle such that the ejected ophthalmic liquid is directed through the at least one opening into the at least one goggle, and
(e) a nebulizing element configured to disperse the ophthalmic liquid in the form of a mist within the at least one eye goggle.

In any of the above described devices, the ejected ophthalmic liquid is preferably directed into the at least one goggle generally in any one of a lateral, medial, superior and inferior direction relative to the surface of the eye. Alternatively and preferably, the ejected ophthalmic liquid is directed into the at least one goggle in a direction substantially different from directly towards the surface of the eye.

In accordance with further preferred embodiments of the present invention, the nebulizing element may be a baffle disposed within the volume of the at least one goggle, such that the ophthalmic liquid impinges on the baffle after ejection, or it may be an element incorporated into the output valve assembly, such that the ophthalmic liquid is dispersed into the mist when passing though the element.

Furthermore, in accordance with yet another preferred embodiment of the present invention, in any of the above described devices, the output valve assembly may be configured to eject a metered dose of the ophthalmic liquid.

In those of the above devices which use a propellant, the propellant may be chosen from a group consisting of compressed air, nitrogen, carbon dioxide, butane, propane, and a mixture of hydrocarbons. Furthermore, in those of the above devices which use a bag-on-valve, the ophthalmic liquid does not come into contact with the propellant. The ophthalmic liquid can thus preferably be used preservative free. The flexible bag preferably comprises a laminated bag.

There is also provided in accordance with a further preferred embodiment of the present invention, a device like those described above, and wherein the at least one eye goggle is a pair of eye goggles, such that the ophthalmic liquid may be applied to both eyes of the subject.

In accordance with still more preferred embodiments of the present invention, there are also provided methods of applying an ophthalmic liquid to at least one eye of a subject, comprising a first step of providing of a dispensing device, the dispensing device comprising:
(a) a flexible bag containing the ophthalmic liquid, the bag having an output valve assembly at an extremity,
(b) an outer container enclosing the flexible bag, the outer container containing a propellant gas under positive pressure,
(c) at least one eye goggle having at least one opening, the outer container being disposed relative to the at least one goggle such that the ejected ophthalmic liquid is directed through the at least one opening into the at least one goggle, and
(d) a nebulizing element such that the ophthalmic liquid is dispersed within the at least one eye goggle in the form of a mist,
   and then the step of activating the valve assembly such that ophthalmic liquid is ejected from the flexible bag into the at least one goggle.

There is further provided, in accordance with still another preferred embodiment of the present invention, a method of applying an ophthalmic liquid to at least one eye of a subject, comprising the step of providing a dispensing device, the dispensing device comprising:
(a) a flexible bag containing the ophthalmic liquid, the bag having an output valve assembly at an extremity,
(b) at least one eye goggle having at least one opening, the outer container being disposed relative to the at least one goggle such that the ejected ophthalmic liquid is directed through the at least one opening into the at least one goggle, and
(c) a nebulizing element configured to disperse the ophthalmic liquid in the form of a mist within the at least one eye goggle,
   and then the step of squeezing the flexible bag such that the output valve assembly is activated and ophthalmic liquid is dispersed in the form of a mist within the at least one goggle.

Finally, in accordance with a further preferred embodiment of the present invention, there is also provided a method of applying an ophthalmic liquid to at least one eye of a subject, comprising firstly the step of providing a dispensing device comprising:
(a) a rigid container containing the ophthalmic liquid and a propellant gas,
(b) an output valve assembly at a first extremity of the rigid container,
(c) a housing in which the rigid container is disposed,
(d) at least one eye goggle having at least one opening in its rim, the housing being disposed relative to the at least one goggle such that ejected ophthalmic liquid is directed through the at least one opening into the at least one goggle,
(e) a nebulizing element configured to disperse the ophthalmic liquid in the form of a mist within the at least one eye goggle,
   and then the step of pressing on a second extremity of the container, such that the output valve is activated and ophthalmic liquid is dispersed in the form of a mist within the at least one goggle.

These and other embodiments of the present invention will become apparent in conjunction with the description and claims that follow.

### BRIEF DESCRIPTION OF THE FIGURES

The preferred embodiments of the invention will hereinafter be described in conjunction with the appended drawings provided to illustrate and not to limit the invention, wherein like designations denote like elements, and in which:

FIG. 1 shows an overall view of one preferred embodiment of the present invention, using a single goggle for treatment of one eye at a time, and a bag-on-valve medicament container;

FIG. 2 schematically illustrates a further preferred embodiment of the present invention, in which a pair of goggles is used to apply the medication to both eyes simultaneously;

FIG. 3 illustrates schematically an alternative and preferred embodiment to that of FIG. 1, in which a conventional, manually pushed, metered-dose, inhalation type of aerosol medicament container is used, adaptable for either the monocular or binocular application described above, and

FIG. 4 illustrates the use of a bag-on-valve embodiment of the present invention adapted for using finger pressure to dispense the medicament into the goggle.

While the invention is susceptible to various modifications and alternative forms, specific embodiments of the invention are provided as examples in the drawings and detailed description. It should be understood that the drawings and detailed description are not intended to limit the invention to the particular form disclosed. Instead, the intention is to cover all modifications, equivalents and alternatives falling within the spirit and scope of the invention as defined by the appended claims.

### DETAILED DESCRIPTION OF THE INVENTION

The present invention relates to a device and method of dispensing medications for treating eye disorders in a subject. The subject is provided with an eye goggle, configured to be fitted around either one of the subject's eyes, and having at least one aperture, preferably in its rim. The ophthalmic liquid is administered to the treated eye through the aperture in a form of a mist produced by nebulizing the liquid using an aerosol type of mechanism.

The device and method of administering a medicament in this form provide safe means of administering the medicament, in that the medicament does not come in fluid communication with a propellant. Furthermore the system and the method of administering a medicament in this form provide accurate dosage volumes, increase the residence time of the medicament in the eye, enhance treatment effectiveness, increase compliance and are convenient to use even by subjects having unsteady hand control.

The unidirectional flow allows for use of preservative-free formulations of the drugs delivered from the container through the system.

According to another preferred embodiment of the present invention, the eye applicator can be in the form of a pair of goggles, such that both eyes can be treated at the same time.

According to preferred embodiments of this invention the goggles enclosing one or both eyes are filled with the produced mist. The eye receives a medication carried in a moist mist having extremely fine cloud like particles contained within the goggle(s). The device and method of this invention can also be applied to treat the outside of the eyes for external eye disease, and to provide intraocular treatment by means of absorption through the cornea, conjunctiva and sclera for other eye disease.

The median diameter of the mist droplet size distribution should preferably be less than about 5 μm, and more preferably, between about 2.5 μm and about 3.5 μm.

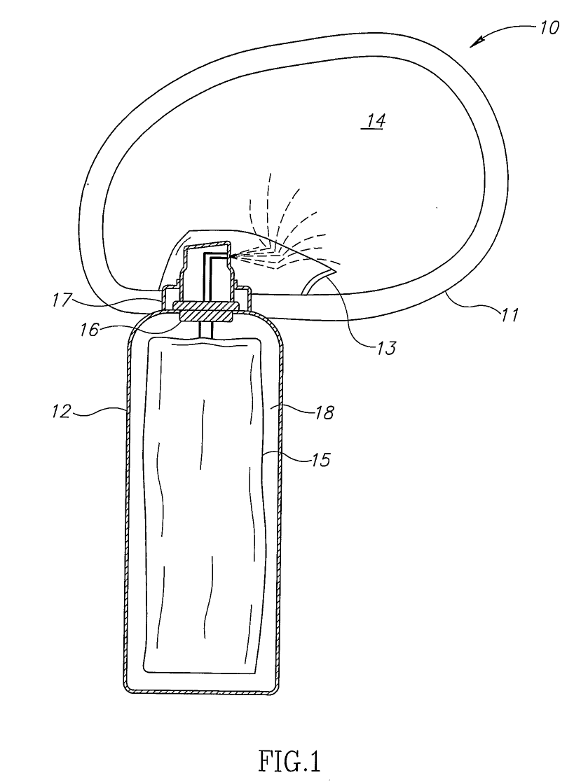

Reference is now made to FIG. 1, which shows an overall schematic view of one preferred embodiment of the present invention, using a single goggle for treatment of one eye at a time. The goggle 10 preferably has a pliant rim 11, such as of rubber or plastic, such that it conforms to the subject's facial profile when applied around the eye to be treated. It thus generates a closed volume around the eye to be treated. The goggle has an aperture or an opening, preferably in its lower, medial or lateral rim, at which the medication dispensing container 12 is attached to the goggle. In general, the opening in the rim is intended to enable entry of the medication into the internal volume of the goggle without obstructing the user's view through the goggle window, and to facilitate entry of the medication in a direction other than straight at the user's eye. However, it is to be understood that the term rim can be understood to apply to any peripheral part of the goggle, and is thuswise used and claimed in this application, and is not meant to be limited to the outer frame of the goggle itself. Opposite and in close proximity to the opening or attachment point, a baffle 13 is optionally positioned such that it breaks up the spray of medication emitted from the medication outlet, and disperses it in a fine mist throughout the inside volume of the goggle 14. The baffle may preferably either be in the form of a continuous sheet, or it may have numerous fine holes. Alternatively and preferably, the outlet nozzle of the valve of the container can be configured such that it emits the medication directly in the form of a mist. It is noted that the mist fills the internal space within the goggle without being directed straight at the front surface of the eye, such that the dispensing operation should not involve any unpleasant feeling for the user.

The goggle is seen to be sealed from all sides (namely, lateral, medial, superior and inferior) to form a chamber so that the mist does not evaporate while residing in the allocated space. The present invention is independent of size and position of such vent on the goggles. Ideally the medication is dispensed within the goggle volume in the form of a mist that surrounds the eye surface. The mouth of such a bag or valve may have one or more holes so as to emit the ingredients in a mist like form into the chamber. The vent and its associated baffle are positioned in such a way so as to enable the mist to spread throughout the enclosed space of the goggle in an even manner.

The medication is preferably contained in a metered dose, bag-on-valve container. Such a container is a “bicompartmental canister” or a “dual chamber canister”, both of which terms are used to refer to a canister that allows the propellant to be kept separately from the ophthalmic liquid. This means that the propellant does not, physically or chemically, react with the ophthalmic liquid, nor does it enter the internal volume of the goggle and thus will not come in contact with the eye. Examples of such canisters are shown in U.S. Pat. Nos.: 5,211,316, 3,581,941 and 3,731,854.

In FIG. 1, there is shown the bag 15 containing the medication. The bag may preferably be an FDA-approved laminated aluminum bag having several layers to ensure safe storage of the medication. Such a bag is suitable for use with liquid drugs, especially those sensitive to air, light or climate-change exposure, and maintains the sterility of the contents. At the top of the bag, there is a hermetically connected valve assembly 16, which is connected to the eye cup (goggle) by means of an adaptor head 17, which can preferably operate as the aerosol actuator. The adapter head 17 may preferably attach the device to the goggle in such a manner that pressure on the base of the device activates the valve assembly to emit a dose of the medicament. The mechanism of the adaptor head and valve assembly schematically shown in FIG. 1 has to be such that the connection of the device to the rim of the goggle does not impede actuation of the valve assembly. FIG. 1 is thus not intended to be a definitive engineering solution of how to arrange the connection of the medication dispensing container to the goggle and how to cause the actuation of the valve assembly, and the claimed invention is not intended to be limited by the arrangement shown in FIG. 1. Thus, for instance, it may also be possible to attach the container just by means of the nozzle itself, but this would require amendment of the generally used aerosol valve and nozzle designs, since the nozzle of aerosols are generally releasably attached to the valve assembly.

The valve assembly can also preferably include a metering valve, in order to dispense a predetermined amount of the medication for each actuation of the aerosol. The propellant gas 18 is compressed in the space between the laminated bag 15 and the outer container 12. There is thus complete separation between the propellant gas and the medication in the laminated bag. Furthermore, the one-way flow operation of the device ensures that there is no back-flow contamination into the medication bag. As a result of both of these features, the need for potentially harmful preservatives in the medication is removed, or limited.

The storage of the medicament under positive pressure in the device of the present invention may also have an advantageous effect for medications containing unstable molecules. The positive pressure may have a stabilizing effect on such molecules.

The bag is filled with a requisite amount of medicament that is required to be dispensed to the user/patient. The valve is fixed to or inserted into the aperture or apertures of the goggle. The valve may incorporate a flexible membrane to enable the medicament to be expelled with a minimum of effort, generally chosen to provide ease of operation even for the elderly or weak. The exit nozzle of the valve may preferably be porous, so that the ingredients are expelled in a shower-like spray to produce a mist within the closed space of the goggle(s) and over the surface of the eye(s). Moreover, the vent is not directed at the eye surface. Again the present invention is independent of the size, position and make of such a valve or bag. Upon the user activating the system, the medicinal ingredients contained in the bag spread evenly in the air surrounding the eye within the internal volume of the goggles. As the spread of medicinal ingredient is noiseless and not jet-like, there is no reflex eye closure and the normal blinking process spreads the optimal amount of medication over the ocular surface.

The propellant may be compressed air, nitrogen, carbon dioxide, butane, propane, or any other suitable gas. According to another embodiment the propellant is a mixture of hydrocarbons. According to this embodiment the hydrocarbons can be butane and propane.

The mass median diameter of the mist droplet size distribution is preferably less than about 5 μm and more preferably between about 2.5 μm and about 3.5 μm. The mist is generated in as noiseless manner as possible.

According to one specific embodiment the outlet and the metering valve are designed to produce fine, cloud like particles forming the mist or fog. Each time the mist is used, a precise measured, predetermined or “metered,” amount of medicine is released, which then comes in contact with the eye.

Ophthalmic liquid can be any pharmaceutical composition designed for the treatment of the eye.

When the actuator is depressed, the metering valve opens and a precisely measured amount of the ophthalmic liquid is released through the outlet into the goggle interior volume. As the dispersing of the ophthalmic liquid is in an even mist form and is noiseless, it does not cause the eye to close and the active ingredients reach and spread across the complete surface of the eye, assisted by normal blinking.

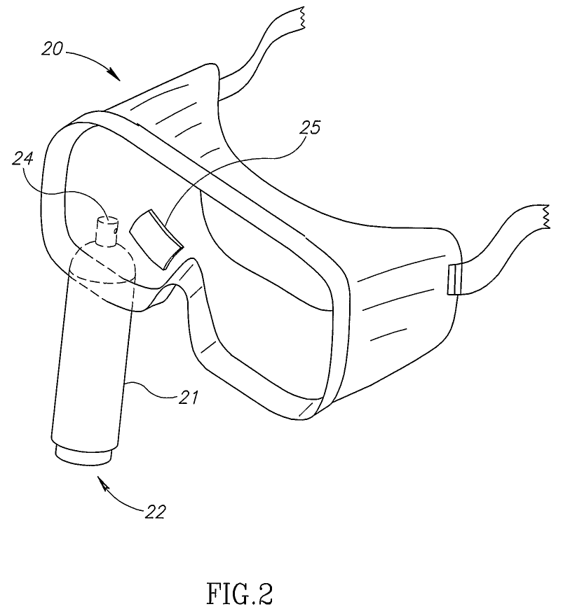

Reference is now made to FIG. 2, which schematically illustrates a further preferred embodiment of the present invention, in which a pair of goggles 20 is used to apply the medication to both eyes simultaneously. One bag-on-valve dispensing device 21 is preferably used to provide medicament mist supply within the whole of the internal volume of the goggles in order to cover both eyes. Alternatively and preferably, one device may be used for each eye. In FIG. 2, there is shown an embodiment wherein the base of the outer container protrudes from an outer housing, and pressure 22 on this base actuates the valve assembly to deliver the dose. The output nozzle 24 and the nebilizing baffle 25 are shown in the goggles.

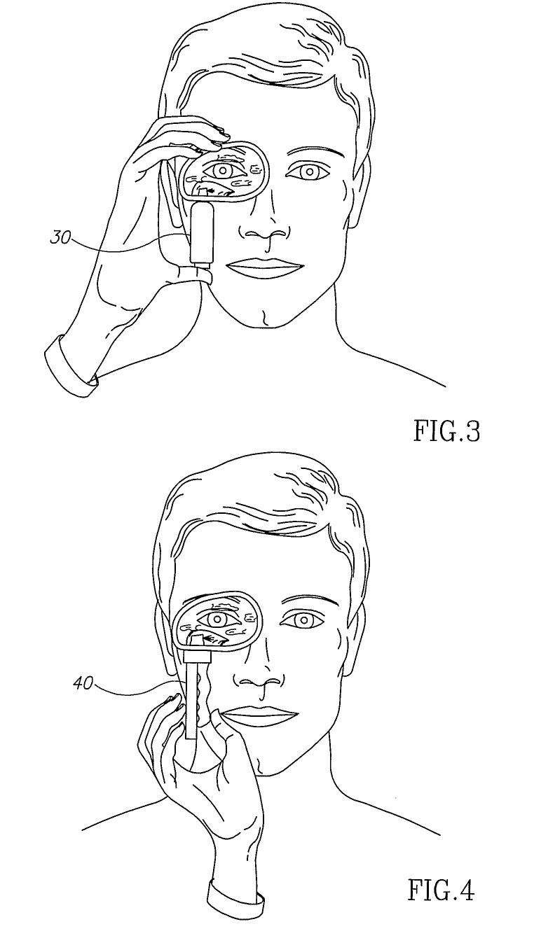

Reference is now made to FIG. 3 which illustrates schematically an alternative and preferred embodiment to that of FIG. 1. In place of the bag-on-valve container, a conventional manually operated, metered-dose, inhalation type of container 30 may be used. Such a container is generally an aerosol, and contains a propellant to enable the medicament to be ejected when the valve is depressed. This embodiment does not therefore enjoy those advantages of the bag-on-valve embodiment of FIG. 1, associated with the complete segregation of medicament and propellant. However, the other advantages of the embodiment of FIG. 1 are also available in the embodiment of FIG. 3. FIG. 3 shows how the device can be held by the user and activated by simple hand pressure. A dose monitoring valve is preferably used for this embodiment, to ensure the dispensing of the correct quantity of medicament.

Reference is now made to FIG. 4 which illustrates schematically a further alternative and preferred embodiment to that of the previous drawings, in which the bag-on-valve 40 is exposed to the user, who simply applies finger pressure in order to expel a dose of medicament from the bag-on-valve.

The present invention is independent of size, position & design of such a specialized goggle/s. The present invention is independent of size, position & design of the apertures in the goggle facet/s. The present invention is independent of size, position & design of the mist generators outlet.

The terms “treat” and “treating” includes preventing, alleviating, ameliorating, halting, restraining, slowing or reversing the progression, or reducing the severity of pathological conditions described above. As such, these methods may include medical therapeutic (acute), prophylactic (prevention) and palliative administration as appropriate.

The term “goggle” or “goggles” is used to refer to any suitable means for confining the mist near one or both eyes. According to the present invention the goggles can be fabricated using any of a variety of suitable materials, including but not limited to plastic, vinyl, composites and the like. The goggles of this invention can also be adapted from pre formed goggles such as diving goggles, swimming goggles, eyecaps, spectacles and so forth. The goggles can be fixed around the eyes using elastic band or ear support members. According to a preferred embodiment the goggles enclose each eye in a separate manner. This allows treating each eye separately.

The propellant is chosen form a group consisting of, but not limited to: compressed air, nitrogen, carbon dioxide, butane, propane etc. However, according to one embodiment, the propellant is compressed air. According to another embodiment the propellant is a mixture of hydrocarbons. According to this embodiment the hydrocarbons can be butane and propane.

While the preferred embodiments of the invention have been illustrated and described, it should be clear that the invention is not limited to these embodiments only. Numerous modifications, changes, variations, substitutions and equivalents will be apparent to those skilled in the art without departing from the spirit and scope of the invention as described in the claims.

1-42. (canceled)

43. A device for applying an ophthalmic liquid to at least one eye of a subject, said device comprising:
a flexible bag containing said ophthalmic liquid;
an output valve assembly at one extremity of said bag;
an outer container enclosing said flexible bag, said outer container containing a propellant gas under positive pressure, such that ophthalmic liquid is ejected from said flexible bag when said valve assembly is activated;
at least one eye goggle having at least one opening in its rim, said outer container being disposed relative to said at least one goggle such that said ejected ophthalmic liquid is directed through said at least one opening into said at least one goggle; and
a nebulizing element configured to disperse said ophthalmic liquid in the form of a mist within said at least one eye goggle.

44. The device according to claim 43, wherein said ejected ophthalmic liquid is directed into said at least one goggle in a direction substantially different from directly towards the surface of said eye, and wherein said direction is generally in any one of a lateral, medial, superior and inferior direction relative to the surface of said eye.

45. The device according to claim 43, wherein said nebulizing element is a baffle disposed within the volume of said at least one goggle, such that said ophthalmic liquid impinges on said baffle after ejection.

46. The device according to claim 43, wherein said nebulizing element is an element incorporated into said output valve assembly, such that said ophthalmic liquid is dispersed into said mist when passing though said element.

47. The device according to claim 43, wherein said output valve assembly is configured to eject a metered dose of said ophthalmic liquid.

48. The device according to claim 43, wherein said propellant is selected from the group consisting of compressed air, nitrogen, carbon dioxide, butane, propane, and a mixture of hydrocarbons.

49. The device according to claim 43, wherein said ophthalmic liquid does not come into contact with said propellant, and wherein said ophthalmic liquid is preservative free.

50. The device according to claim 43, wherein said flexible bag comprises a laminated bag.

51. The device according to claim 43, wherein said at least one eye goggle is a pair of eye goggles, such that said ophthalmic liquid may be applied to both eyes of the subject.

52. The device according to claim 43, wherein said flexible bag is configured such that when squeezed, a dose of said ophthalmic liquid is dispersed in the form of a mist within said at least one goggle.

53. A device for applying an ophthalmic liquid to the eye of a subject, comprising:
a rigid container containing said ophthalmic liquid and a propellant gas;
an output valve at a first extremity of said rigid container;
a housing in which said rigid container is disposed, such that pressure on a second extremity of said container activates said output valve and ejects ophthalmic liquid from said rigid container;
at least one eye goggle having at least one opening in its rim, said housing being disposed relative to said at least one goggle such that said ejected ophthalmic liquid is directed through said at least one opening into said at least one goggle; and
a nebulizing element configured to disperse said ophthalmic liquid in the form of a mist within said at least one eye goggle.

54. The device according to claim 53, wherein said ejected ophthalmic liquid is directed into said at least one goggle in a direction substantially different from directly towards the surface of said eye, and wherein said direction is generally in any one of a lateral, medial, superior and inferior direction relative to the surface of said eye.

55. The device according to claim 53, wherein said nebulizing element is a baffle disposed within the volume of said at least one goggle, such that said ophthalmic liquid impinges on said baffle after ejection.

56. The device according to claim 53, wherein said nebulizing element is an element incorporated into said output valve assembly, such that said ophthalmic liquid is dispersed into said mist when passing though said element.

57. The device according to claim 53, wherein said output valve assembly is configured to eject a metered dose of said ophthalmic liquid.

58. The device according to claim 53, wherein said ophthalmic liquid is preservative free.

59. The device according to claim 53, wherein said flexible bag comprises a laminated bag.

60. A device according to claim 53, wherein said at least one eye goggle is a pair of eye goggles, such that said ophthalmic liquid may be applied to both eyes of the subject.

61. A method of applying an ophthalmic liquid to at least one eye of a subject, the method comprising:
providing a dispensing device comprising:
a flexible bag containing said ophthalmic liquid, said bag having an output valve assembly at an extremity;
an outer container enclosing said flexible bag, said outer container containing a propellant gas under positive pressure;
at least one eye goggle having at least one opening, said outer container being disposed relative to said at least one goggle such that said ejected ophthalmic liquid is directed through said at least one opening into said at least one goggle; and
a nebulizing element such that said ophthalmic liquid is dispersed within said at least one eye goggle in the form of a mist; and
activating said valve assembly such that ophthalmic liquid is ejected from said flexible bag into said at least one goggle.

62. The method of applying an ophthalmic liquid according to claim 61, wherein said ophthalmic liquid is preservative free.

63. The method of applying an ophthalmic liquid according to claim 61, wherein said ejected ophthalmic liquid is directed into said at least one goggle in a direction substantially different from a normal to directly towards the surface of said eye, and wherein said direction is generally in any one of a lateral, medial, superior and inferior direction relative to the surface of said eye.

64. The method of applying an ophthalmic liquid according to claim 61, wherein said nebulizing element is a baffle disposed within the volume of said at least one goggle, such that said ophthalmic liquid impinges on said baffle after ejection.

65. The method of applying an ophthalmic liquid according to claim 61, wherein said nebulizing element is an element incorporated into said output valve assembly, such that said ophthalmic liquid is dispersed into said mist when passing though said element.

66. The method of applying an ophthalmic liquid according to claim 61, wherein said output valve assembly is configured to eject a metered dose of said ophthalmic liquid.

67. The method of applying an ophthalmic liquid according to claim 61, wherein said propellant is selected from the group consisting of compressed air, nitrogen, carbon dioxide, butane, propane, and a mixture of hydrocarbons.

68. The method of applying an ophthalmic liquid according to claim 61, wherein said ophthalmic liquid does not come into contact with said propellant.

69. The method of applying an ophthalmic liquid according to claim 61, wherein said flexible bag comprises a laminated bag.

70. The method of applying an ophthalmic liquid according to claim 61, wherein said at least one eye goggle is a pair of eye goggles, such that said ophthalmic liquid may be applied to both eyes of the subject.

71. The method of applying an ophthalmic liquid according to claim 61, wherein activating said valve assembly comprises squeezing said flexible bag such that said output valve assembly is activated and ophthalmic liquid is dispersed in the form of a mist within said at least one goggle.

72. A method of applying an ophthalmic liquid to at least one eye of a subject, the method comprising:
providing a dispensing device according to claim 53, and
pressing on said second extremity of said container, such that said output valve is activated and ophthalmic liquid is dispersed in the form of a mist within said at least one goggle.
